(19) United States
(12) Patent Application Publication
(10) Pub. No.: US 2013/0331921 A1
(43) Pub. Date: Dec. 12, 2013

(54) BIFURCATED CATHETER

(71) Applicant: Gary Roubin, New York, NY (US)

(72) Inventor: Gary Roubin, New York, NY (US)

(21) Appl. No.: 13/914,430

(22) Filed: Jun. 10, 2013

Related U.S. Application Data
(60) Provisional application No. 61/657,427, filed on Jun. 8, 2012.

(51) Int. Cl. A61N 1/05

(52) U.S. Cl. CPC A61N 1/0587
USPC 607/122

(57) ABSTRACT

Systems and methods are disclosed for a bifurcated catheter, and more particularly, to denervation of arterial nerves using a bifurcated catheter. In embodiments, the catheter comprises, for example, a catheter shaft bifurcated into a first catheter energy member and a second catheter energy member, wherein the first catheter energy member comprises a first transducer and the second catheter energy member comprises a second transducer, and wherein the first and second transducers are configured to cause denervation of an arterial nerve of the patient.

### BACKGROUND

1. Field of the Technology

The present technology relates generally to a bifurcated catheter, and more particularly, to denervation of arterial nerves using a bifurcated catheter.

2. Related Art

Hypertension, or high blood pressure, is a common medical condition where the blood pressure in the arteries is elevated, requiring the heart to work harder than normal to circulate blood through its surrounding blood vessels. Many people with high blood pressure may improve their condition using concurrent use of antihypertensive drugs, drugs used especially for the treatment of hypertension. However, some patients have the medical condition of resistant hypertension, which is hypertension that remains above goal blood pressure levels in spite of antihypertensive drug treatment.

### SUMMARY

In one aspect, there is provided a device configured to be inserted into an artery of a patient, the device comprising: a catheter shaft bifurcated into a first catheter energy member and a second catheter energy member, wherein the first catheter energy member comprises a first transducer and the second catheter energy member comprises a second transducer, and wherein the first and second transducers are configured to cause denervation of an arterial nerve of the patient.

In another aspect, there is provided a catheter, comprising: a first catheter energy member comprising at least one transducer, wherein the first catheter energy member is configured to be deployed in an internal carotid artery; a second catheter energy member comprising at least one transducer, wherein the second catheter energy member is configured to be deployed in an external carotid artery; wherein the transducer of the first catheter energy member and the transducer of the second catheter energy member are configured to emit energy to a carotid sinus.

In another aspect, there is provided a bifurcated catheter deployment kit comprising: a sheath configured to be deployed into a common carotid of a patient; a first guidewire; a second guidewire; and a bifurcated catheter, deployed into a carotid artery of the patient via the sheath, the bifurcated catheter comprising: a first catheter energy member comprising at least one transducer, wherein the first catheter energy member is configured to be deployed, via the first guidewire, into an internal carotid artery; a second catheter energy member comprising at least one transducer, wherein the second catheter energy member is configured to be deployed, via the second guidewire, into an external carotid artery.

### BRIEF DESCRIPTION OF THE DRAWINGS

Embodiments of the present technology are described below with reference to the attached drawings, in which:

FIG. 1A illustrates a schematic view of an exemplary set of carotid arteries;

FIG. 1B illustrates a schematic view of an exemplary set of internal and external carotid arteries bifurcated from a common carotid artery;

FIG. 2 illustrates a schematic view of a bifurcated catheter system, according to embodiments of the present technology;

FIG. 3 illustrates a schematic view of a bifurcated catheter inside a carotid sheath, according to embodiments of the present technology;

FIG. 4A illustrates a bifurcated catheter in its preferred position within the carotid arteries for denervation of the carotid sinus, according to embodiments of the present technology;

FIG. 4B illustrates a bifurcated catheter in its preferred position within the carotid arteries for denervation of the carotid sinus, according to embodiments of the present technology; and

FIG. 5 illustrates is a flow chart showing the steps for deployment and application of a bifurcated catheter, according to embodiments of the present technology.

### DETAILED DESCRIPTION

Aspects and embodiments of the present technology are directed to a bifurcated catheter for insertion into each distal branch of a branched or bifurcated body lumen such as the blood vessels of the cardiovascular system, the lymph vessels of the lymphatic system, the bronchi of the respiratory tract, the ureters of the renal system, etc. The catheter is configured to perform in each distal branch a therapeutic function such as delivery of fluid, energy or components, retrieval of body fluid or tissue, monitoring, imaging, etc.

Aspects of the disclosed technology are described below with reference to a particular embodiment configured to denervate the arterial nerves and/or the baroreceptors of the carotid sinus. The inventor discovered that some patients who have undergone carotid endarterectomy have experienced a permanent reduction on blood pressure. Careful review of many patients over considerable time revealed to the inventor that at least in some such circumstances such reduction in blood pressure was likely due to trauma of the arterial nerve and/or baroreceptors. The inventor developed the disclosed bifurcated catheter and associated components and methodologies so that a minimally invasive procedure may be used to achieve a permanent reduction in blood pressure. Such a procedure may advantageously be used, for example, with patients having elevated blood pressure that is not responsive to conventional drug therapy, or as a preventive therapy for patients who will develop high blood pressure in the future.

Specifically, to denervate the arterial nerves and/or the baroreceptors of the carotid sinus, the bifurcated catheter of the present technology is described with respect to being used in one set of arteries, namely the carotid arteries (including the common carotid arteries, external carotid arteries and internal carotid arteries). It should be appreciated, however, that embodiments of the present technology may be implemented in other blood vessels that include a bifurcation of one vessel into two vessels.

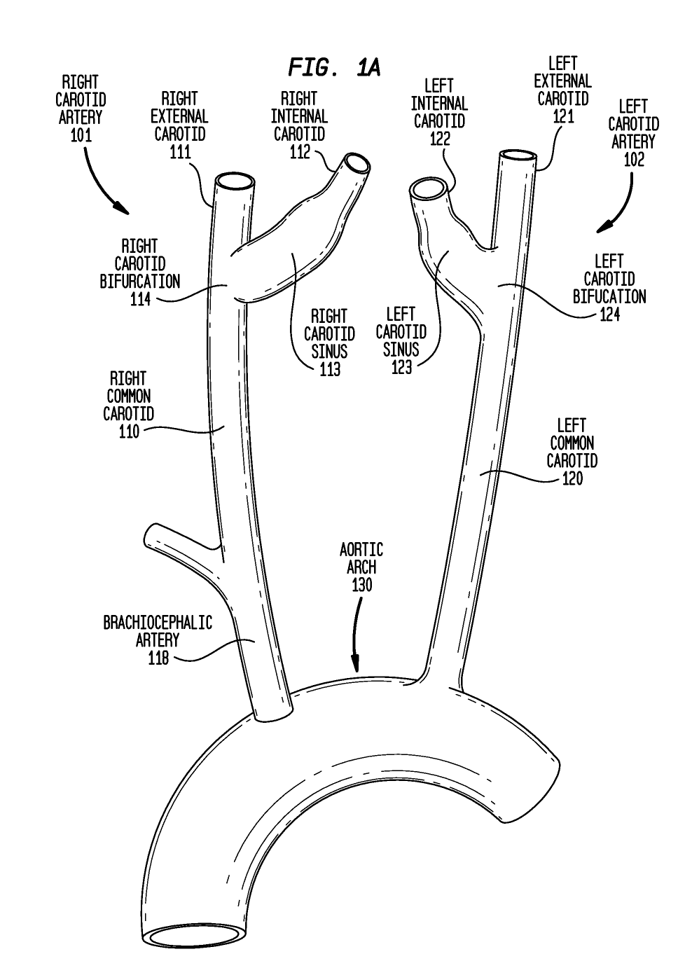

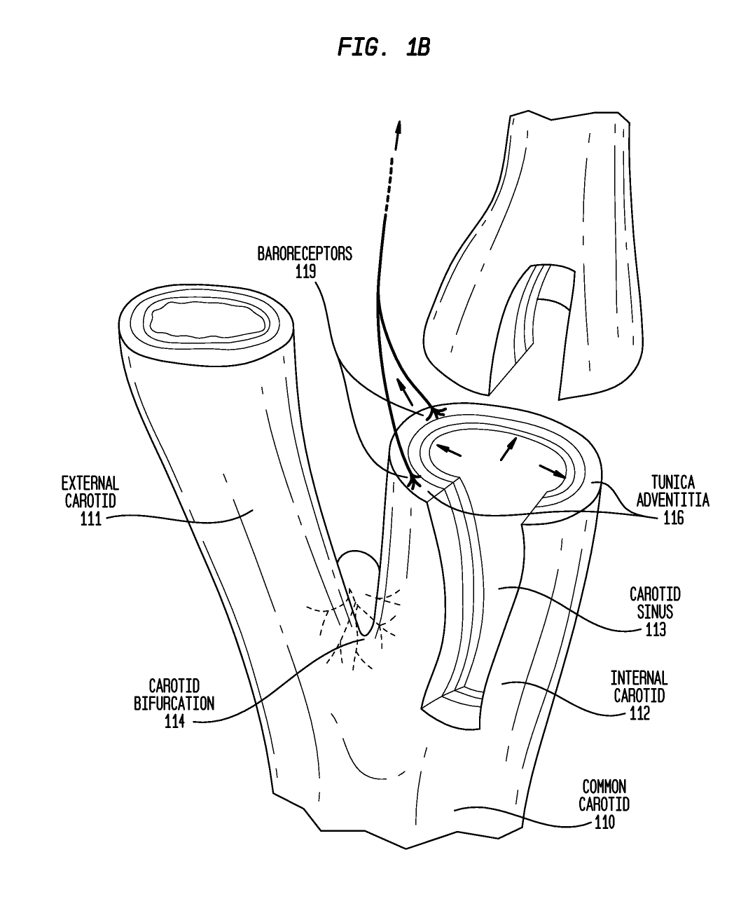

The carotid arteries are blood vessels that supply blood to the head, neck and brain. One carotid artery is positioned on each side of the neck (hereinafter, the “left” and “right” sides). FIG. 1A is a schematic view of the carotid arteries. The right carotid artery 101 includes a proximal vessel referred to as the right common carotid 110, that is divided into two distal branches: the right external carotid 111 and the right internal carotid 112 at bifurcation 114. The left carotid artery 102 includes left common carotid 120, left external carotid 121 and left internal carotid 122. The right common carotid artery branches from the brachiocephalic artery 118 and extends up the right side of the neck. The left common carotid artery branches from the arch of the aorta 130 and extends up the left side of the neck.

As noted, each of the left carotid artery and right carotid artery include external and internal carotid arteries. The common carotid on each side of the neck bifurcates, and branches into the external and internal carotid arteries. The internal carotid arteries supply the brain (not shown) with blood, while the external carotid nourishes other portions of the head, such as the face, scalp, skull and meninges (not shown). For example, as shown in FIG. 1A, right carotid artery 101 bifurcates at right carotid bifurcation 114 and left carotid artery 102 bifurcates at left carotid bifurcation 124.

FIG. 1B shows a close-up schematic view of right carotid artery 101, including right common carotid 110, right external carotid 111 and right internal carotid 112 as shown in FIG. 1A. The artery 101 also includes a carotid sinus 113, as shown in FIG. 1B. Carotid sinus 113 is the dilated area in the internal carotid and/or bifurcation of common carotid 110, carotid bifurcation 112 (the carotid sinus may also, in some people, extend into the common carotid). The carotid sinus is sensitive to pressure changes in the blood traveling through the carotid arteries. More specifically, the carotid sinus contains numerous baroreceptors 119 which function as a sampling area for several homeostatic mechanisms that contribute to maintaining blood pressure. Although FIG. 1B is shown as having two sets of baroreceptors 119, tunica adventitia 116 may include many more baroreceptors throughout its arterial layer. In other words, the carotid sinus and its baroreceptors are sensitive to changes in blood pressure and help the body to maintain blood pressure stability. Arterial baroreceptors are stretch receptors that are stimulated by distortion of the arterial wall when pressure changes. As shown in FIG. 1B, arterial baroreceptors 119 are located in the outermost layer, the tunica adventitia (shown as element 116 in FIG. 1B), of the carotid artery. In fact, baroreceptors can identify changes in blood pressure (both the average blood pressure and the rate of change in pressure) with each palpation or “firing” of the carotid artery. If blood pressure falls, baroreceptor firing rate decreases and the baroreceptors react to help restore blood pressure to normal levels by increasing heart rate. Therefore, the carotid sinus, and the baroreceptors in the walls of the sinus, is discovered by the inventor to have effective control over a person's blood pressure, and the carotid sinus may be controlled to change the carotid sinus's effect on that blood pressure. As such, denervation of the carotid sinus may effectively reduce blood pressure by not allowing its baroreceptors to give notice of changes in blood pressure to the brain or restore reduced blood pressure. For example, denervation of the carotid sinus may reduce a patient's blood pressure by 20 points or more.

Although, as noted, it has been known to cause denervation of renal arteries to reduce blood pressure, the carotid sinus is more sensitive to changes in blood pressure than renal arteries. Therefore, denervation of the carotid sinus can have a sharper effect on lowering blood pressure than methods affecting the renal arteries.

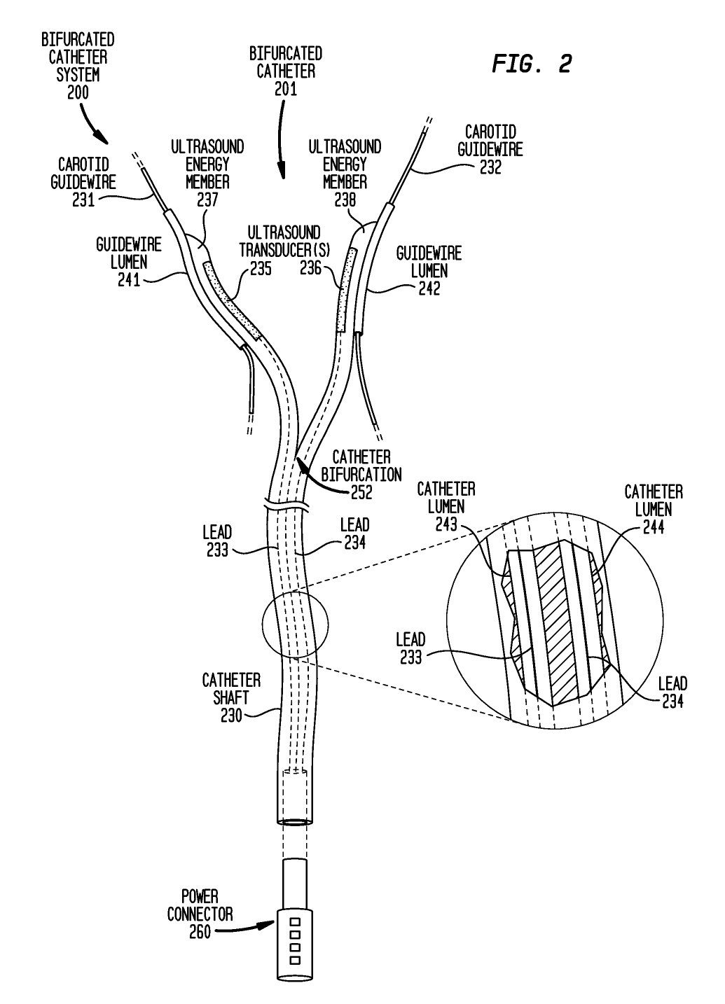

The bifurcated catheter system of the present technology includes a catheter having a common shaft that is bifurcated into two branches. The bifurcated branches of the bifurcated catheter system are inserted into the bifurcated or branched portions of a bifurcated body lumen. An example of such a bifurcated catheter system of the present technology is shown in FIG. 2, which shows bifurcated catheter system 200 configured to be inserted into the carotid arteries. Bifurcated catheter system 200 includes a catheter 201 having a catheter shaft 230 that is bifurcated into two branches, ultrasound energy members 237 and 238. Ultrasound energy member 237 is configured to extend into, for example, internal carotid 112 or external carotid 111. Ultrasound energy member 238 is configured to extend into whichever of internal carotid 112 or external carotid 111 that ultrasound energy member 237 did not extend into. Bifurcated catheter 201 delivers energy, such as ultrasound, to baroreceptors and other nerves in the artery to cause denervation of the arterial nerves.

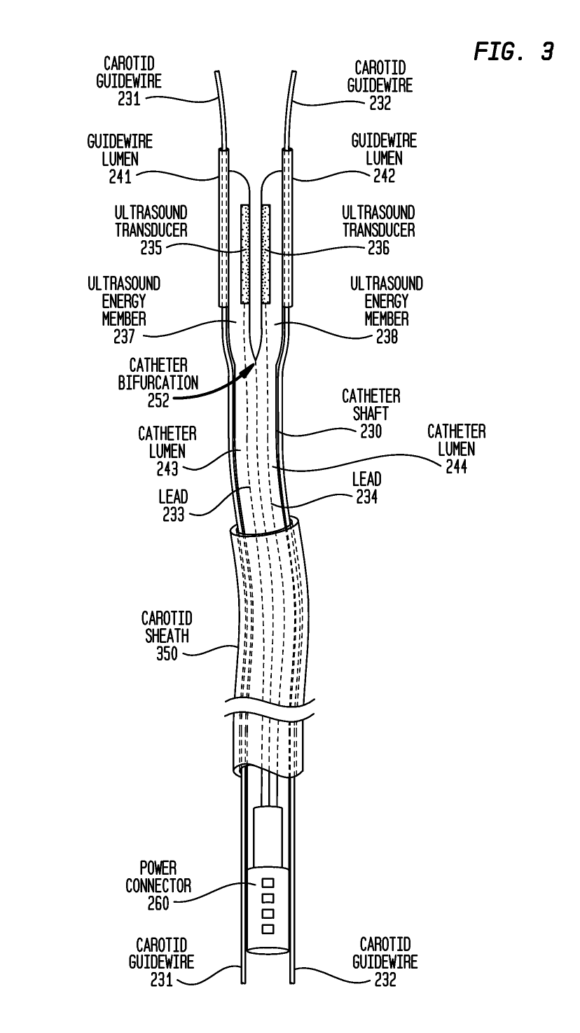

The bifurcated catheter system 200 of FIG. 2 also includes guidewire lumen 241 and 242 and carotid guidewire 231 and 232. Guidewire 231 and 232 extend through guidewire lumen 241 and 242, respectively. Guidewires 231 and 232 and guidewire lumens 241 and 242 are used, along with a sheath (such as, for example, carotid sheath 350, shown in FIG. 3), to deploy bifurcated catheter 201 into the carotid arteries. This process will be described in more detail below with respect to FIG. 3.

As shown in FIG. 2, bifurcated catheter 201 includes at least two lumens, such as catheter lumen 243 that runs from the proximal end of catheter shaft 230 up to or including the distal end of ultrasound energy member 237, and catheter lumen 244 that runs from the proximal end of catheter shaft 230 up to or including the distal end of ultrasound energy member 238. Although this embodiment of the present technology is described with two catheter lumens, catheter shaft 230 may also only include one lumen that bifurcates into two lumens at catheter bifurcation 252. Bifurcated catheter 201 may also have more than two lumens. Catheter lumens 243 and 244 may have a consistent diameter throughout their length, or may vary in diameter as necessary to accommodate leads 233 and 234 running through them. As referred to herein, the term “distal” end when used with respect to a component of the bifurcated catheter system means the end towards the bifurcated portion of the catheter, and the term “proximal” end when used with respect to a component of the bifurcated catheter system means the end opposite the bifurcated portion, or the end of the catheter shaft farthest away from the bifurcation, of the catheter.

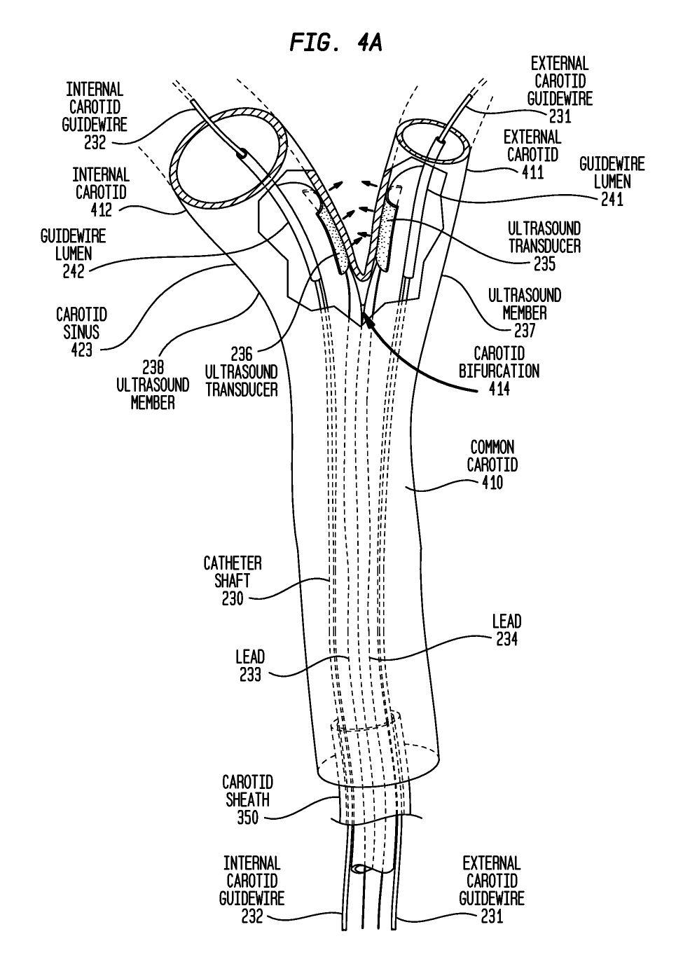

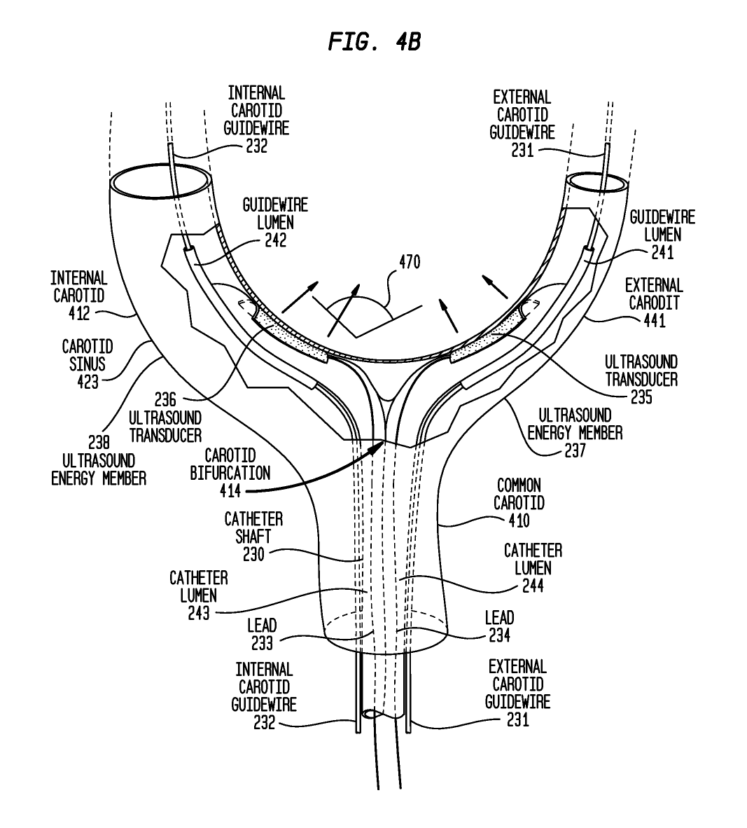

The bifurcated catheter 201 in FIG. 2 also includes transducer(s) 235 located within or on the surface of ultrasound energy member 237, and transducer(s) 236 located within or on the surface of ultrasound energy member 238. Transducers 235 and 236 provide ultrasound energy to the baroreceptors and other nerves in the carotid arteries. Lead 233 is fed through catheter lumen 243 and extends from the proximal end of catheter shaft 230 and through ultrasound energy member 237. The distal end of lead 233 is connected to ultrasound transducer 235. Lead 234 is fed through catheter lumen 244 and extends from the proximal end of catheter shaft 230 and through ultrasound energy member 238. The distal end of lead 234 is connected to ultrasound transducer 236. As will be described further below with respect to FIGS. 4A and 4B, leads 233 and 234 provide energy through catheter shaft 230 and through the bifurcated portions of bifurcated catheter 201, ultrasound energy members 237 and 238, to ultrasound transducers 235 and 236. Ultrasound energy members 237 and 238 are flexible such that they may track the location and movement of leads 233 and 234 running through them.

It should be appreciated that although embodiments of the present technology are discussed herein with respect to using “ultrasound transducers” and “ultrasound energy members” and with respect to emitting ultrasound energy, various other types of energy may be used. For example, it should be appreciated that embodiments of the present technology may be implemented using thermal energy, microwave energy, radio frequency, and other types of energy.

Power connector 260 is connected to the proximal end of catheter shaft 230 and provides power/energy to leads 233 and 234. Therefore, the proximal end of leads 233 and 234 may exit the proximal end of catheter shaft 230 from catheter lumens 243 and 244, respectively, and connect to power connector 260. Power connector 260 may also provide a connection to the exterior of the patient, either for external power or to connect to a user interface (UI) for a surgeon or other medical professional to provide control over the bifurcated catheter and its ultrasound emitting transducers. For example, power connector 260 may connect to a console outside the body of the patient so that the doctor may control how the transducers are energized, at what levels the transducers emit energy, and various other aspects of the catheter system.

FIG. 3 illustrates a schematic view of a bifurcated catheter system 300, in which embodiments of the present technology may be implemented. Bifurcated catheter system 300 includes carotid sheath 350. For example, carotid sheath 350 may have a 6 French-7 French diameter. However, sheaths may be used depending on the application and size of catheter. Carotid sheath 350, in conjunction with carotid guidewires 231 and 232, is used for insertion and placement of the catheter into the carotid arteries. Guidewires 231 and 232 may be standard 0.014″ coronary/carotid guidewires typically used, for example, carotid interventions. However, guidewires 231 and 232 may also be slightly larger such as 0.018-0.025″ small vessel guidewires, or may be of a different size. The size of the guidewires used depends on the size of catheter used. More specifically, the sheath is inserted into the common carotid, such as, for example, common carotid 110 or 120, which are shown FIG. 1A, using well-known conventional techniques. For example, to place the sheath in to the common carotid, a sheath deployment guidewire (not shown) may be inserted into the groin of the patient and through patient arteries into the common carotid, and the sheath is fed over that deployment guidewire into the common carotid. However, the sheath is inserted into the common carotid such that the proximal end of the sheath remains outside the patient's body.

After sheath 350 is inserted into the common carotid, guidewires 231 and 232 are, one by one, inserted into the proximal end of the sheath. Each guidewire is fed through the sheath such that the distal end of the guidewire exits the distal end of the sheath and into either the external or the internal carotid artery. One guidewire, such as guidewire 231, must be inserted into the exterior carotid and the other guidewire, such as guidewire 232, must be inserted into the internal carotid. Therefore, the first guidewire to be inserted may be inserted until it enters either the internal or exterior carotid artery, and the second guidewire inserted may be tailored such that it is directed to a carotid artery of choice, for example so that it enters whichever bifurcated portion of the carotid artery that the first guidewire did not enter. For example, the distal end/tip of the second guidewire may be bent or angled so that it enters the required bifurcated portion of the carotid artery.

The proximal ends of guidewires 231 and 232, similar to the proximal end of sheath 350, remain outside the patient's body after insertion. Before insertion, guidewires 231 and 232 may be marked or labeled on, for example, their proximal end before insertion so that the user records which guidewire is inserted into which carotid artery.

Although embodiments of bifurcated catheter may be symmetrical (such that either ultrasound energy member may be inserted into either the external or internal carotid artery), in other embodiments, the ultrasound energy members of the bifurcated catheter are asymmetrical, each being configured to be inserted into its corresponding distal branch of the branched body lumen. In such an asymmetrical bifurcated catheter, the ultrasound energy members may be specifically tailored or constructed for either the external or internal carotid artery, requiring specific placement of that energy member in its respective carotid artery.

After the guidewires are inserted into their respective bifurcated portions of the carotid artery, bifurcated catheter 201 is inserted into the patient using guidewires 231 and 232 and sheath 350, which are in place within the patient.

First, the proximal ends of guidewires 231 and 232 are fed through the distal ends of guidewire lumens 241 and 242, respectively. Guidewire lumens 241 and 242 are attached to the sides of ultrasound energy members 237 and 238, respectively. For example, guidewire lumens 241 and 242 may be glued to their respective ultrasound energy members using adhesive, or may be connected in other conventional techniques of attaching medical device components.

Furthermore, in one embodiment, guidewire lumens 241 and 242 are shorter than their respective ultrasound energy members (e.g. rapid exchange guidewire system). The distal ends of guidewire lumens 241 and 242 extend beyond the respective distal ends of ultrasound energy members 237 and 238, but only extend a portion of their respective ultrasound energy members towards their proximal ends. For example, the length of the guidewires may be as little as 15, 10, 5, or even shorter, while the catheter may be over 100 cm long. However, the proximal ends of guidewire lumens 241 and 242 may extend beyond the respective proximal ends of ultrasound energy members 237 and 238 without changing the functionality of the bifurcated catheter 201. For example, a full over-the-wire guidewire system, or other guidewire systems, is also applicable to the present technology.

After the proximal ends of the guidewires are fed through the distal ends of their respective guidewire lumens, bifurcated catheter 201 is fed along the guidewires and through the sheath until catheter 201 reaches the common carotid. As shown in FIG. 3, the two bifurcated portions of bifurcated catheter system 300 (including ultrasound energy members 237 and 238 and their associated components, are collapsed together to travel as a single entity until reaching the carotid artery bifurcation. After the distal end of bifurcated catheter 201, and therefore the distal ends of ultrasound energy members 237 and 238, reach the bifurcation in the carotid artery (such as, for example, bifurcation 114 or 124 as shown in FIG. 1A), ultrasound energy member 237 will continue to follow its associated carotid guidewire into the guidewire's associated internal or external carotid artery. In other words, if, upon insertion, the distal end of carotid guidewire 231 was fed into external carotid 111 (as shown in FIG. 1A) and the proximal end of guidewire 231 was fed into guidewire lumen 241 (as shown in FIG. 2, for example), then ultrasound energy member 237 would follow guidewire 231 into external carotid 111. Ultrasound energy members 237 and 238 are inserted into their respective internal or external carotid arteries until the bifurcation of bifurcated catheter 201, catheter bifurcation 252, reaches the bifurcation of the carotid artery. At that time, the internal wall of bifurcated catheter 201 will press up against the artery bifurcation and prevent further progress of catheter 201 being inserted. The engagement of the catheter bifurcation 252 and the carotid bifurcation allows ultrasound energy members 237 and 238, and therefore ultrasound transducers 235 and 236, to surround the carotid bifurcation and carotid sinus from within the carotid artery. The relationship between ultrasound energy members 237 and 238, ultrasound transducers 235 and 236, the carotid bifurcation and carotid sinus will be discussed further below with respect to FIGS. 4A and 4B.

FIG. 4A shows bifurcated catheter 201 in its operable position within the carotid arteries for denervation of the carotid sinus. As noted, ultrasound energy members 237 and 238 are located in their respective internal or external carotid arteries until the bifurcation of bifurcated catheter 201 presses against the bifurcation of the carotid artery and surround the carotid bifurcation and carotid sinus from within the carotid artery. As shown in FIG. 4A, when in the desired location within carotid arteries 411 and 412, ultrasound transducers 235 and 236 are located on the inside portions of their respective internal and external carotid arteries. In other words, ultrasound transducer 235 is located on a portion of ultrasound energy member 237 that is closest to the portion of external carotid 411 that is adjacent to internal carotid 412, and ultrasound transducer 236 is located on a portion of ultrasound energy member 238 that is closest to the portion of internal carotid 412 that is adjacent to external carotid 411. Furthermore, ultrasound transducers 235 and 236 are located adjacent to carotid sinus 423. As noted, carotid sinus 423 is the dilated area in internal carotid 412 (and, in some people, common carotid 410) close to carotid bifurcation 414. Ultrasound transducers 235 and 236 emit ultrasound waves through the walls of external carotid 411 and internal carotid 412 (and, if desired, common carotid 410) into carotid sinus 423 and its surrounding nerves and tissue. Emitting ultrasound waves into carotid sinus 423 causes denervation of the carotid sinus, disabling the carotid sinus and other nerves adjacent to the sinus. More specifically, denervation disables afferent nerves that connect to the medulla oblongata and other afferent pathways to the cardiovascular control centers in the brainstem. As noted, denervation of the carotid sinus may effectively reduce blood pressure.

FIG. 4B is similar to FIG. 4A in structure and application, except that the bifurcation of common carotid 410 into external carotid 411 and internal carotid 412 spans an angle (the angle between the proximal ends of external carotid 411 and internal carotid 412) that is greater than the corresponding angle of the bifurcation in FIG. 4A. As shown in FIG. 4B, bifurcated catheter 401 may fit a wide range of bifurcation shapes or angles. For example, bifurcated catheter may fit a carotid bifurcation as narrow as 15 degrees or as wide as 90 degrees or greater.

Ultrasound transducers 235 and 236 as shown in FIGS. 4A and 4B are shown as single elongate transducers in the form of convex panels that extend along portions of internal and external carotid arteries. However, embodiments of the present technology may be implemented using two transducers on each artery, using transducer arrays, including many different transducers on each artery, or using any of other known combinations/techniques of transducers. For example, if implemented using a transducer array including multiple transducers on each of the internal and external carotid arteries, the transducers on each artery may be lined up to match a transducer on the opposing artery (so that, for example, each artery includes a transducer the same distance away from the carotid bifurcation as a transducer on the opposing artery), the transducers may be staggered so that no arteries line up with a transducer on the opposing artery, or any other transducer alignment. Transducers may also be grouped in pairs or other combinations of transducers. Furthermore, the transducers may be curved to direct the ultrasound energy uniformly across portions of the carotid arteries, or positioned to target distribution of the ultrasound energy to specific portions of the arteries and carotid sinus. For example, transducers may be distributed such that they target certain nerves or groups of nerves within the carotid sinus. In other words, the different patterns of transducers allows for different applications of constructive interference with the carotid sinus, and allows the user to direct energy to certain groups of nerves at different levels.

Furthermore, transducers may be placed on portions of the bifurcated catheter other than on energy members 237 and 238. For example, catheter bifurcation 252 may include one or more transducers. Placing transducers on catheter bifurcation 242, as well as on energy members 237 and 238, would allow for more transducer coverage of the carotid sinus, and would allow the catheter to emit energy to the carotid sinus for denervation from even more directions. More specifically, for example, such combination of transducers would allow for transducers to at least partially surround the carotid sinus.

Furthermore, the ultrasound transducers, such as transducers 235 and 236, may be individually powered so that the amount of power transmitted to and by each transducer is individually controlled. For example, as shown in FIGS. 2-4B, catheter lumen 243 and 244 are shown as including a single lead connected to a single transducer located in each ultrasound energy member 237 and 238. However, multiple leads may be included in the catheter system, either in each of the single catheter lumen or across multiple catheter lumen. Each of the multiple leads may then be connected to different transducers, which, as noted, may be situated on ultrasound energy members 237 and 238 in an array, in groups, or in any other configuration. Connecting each transducer to separate leads allows each to be powered separately, which allows the system to achieve a balanced and deeply penetrating ultrasonic beam. For example, the transducers may emit a large amount of energy to cause complete denervation of the carotid sinus and other surrounding nerves. On the other hand, if desired, the transducers may be controlled to emit a lesser amount of energy to cause only partial denervation of the nerve(s).

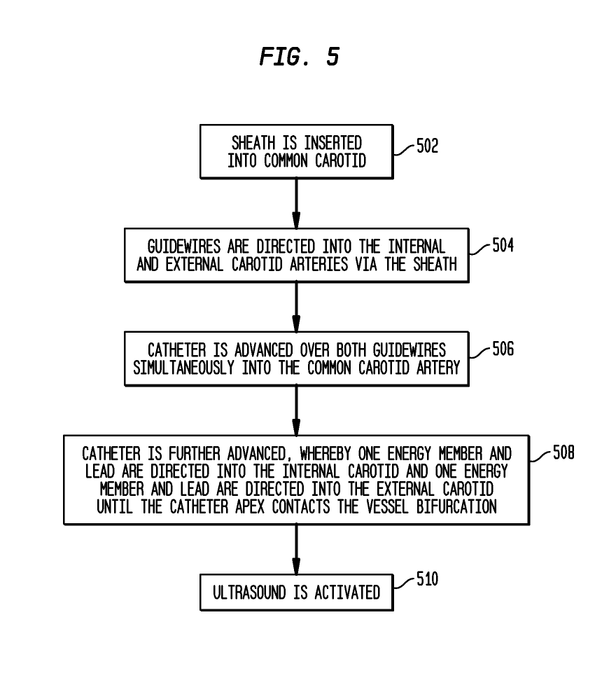

FIG. 5 is a flow chart showing the steps for deployment and application of the bifurcated catheter, according to embodiments of the present technology. As noted in block 501, the carotid sheath is inserted into the common carotid, such as, for example, common carotid 110 or 120, which are shown FIG. 1A, using well-known conventional techniques. As noted in block 502, guidewires are then, one by one, inserted into the proximal end of the sheath. Each guidewire is fed through the sheath such that the distal end of the guidewire exits the distal end of the sheath and into either the external or the internal carotid artery. As noted in block 503, the bifurcated catheter, as described in FIG. 2, is advanced over the guidewires into the common carotid artery. As noted in block 504, the catheter is then advanced whereby one energy member (and therefore the lead located in the energy member) is directed into the internal carotid and the other energy member (and therefore the other lead located in that energy member) is directed into the external carotid until the catheter bifurcation contacts the carotid artery bifurcation. When the transducers on the energy members are situated in their desired positions, the ultrasound is activated so that the transducers emit ultrasound energy to the carotid sinus for denervation.

The technology described and claimed herein is not to be limited in scope by the specific preferred embodiments herein disclosed, since these embodiments are intended as illustrations, and not limitations, of several aspects of the technology. Any equivalent embodiments are intended to be within the scope of this technology. Indeed, various modifications of the technology in addition to those shown and described herein will become apparent to those skilled in the art from the foregoing description. Such modifications are also intended to fall within the scope of the appended claims.

What is claimed is:

1. A device configured to be inserted into an artery of a patient, the device comprising:
a catheter shaft bifurcated into a first catheter energy member and a second catheter energy member,
wherein the first catheter energy member comprises a first transducer and the second catheter energy member comprises a second transducer, and
wherein the first and second transducers are configured to cause denervation of an arterial nerve of the patient.

2. The device of claim 1, wherein the first catheter energy member is configured to be deployed in an internal carotid artery.

3. The device of claim 2, wherein the second catheter energy member is configured to be deployed in an external carotid artery.

4. The device of claim 3, wherein the first transducer is configured to emit energy in a direction from the internal carotid artery towards an arterial nerve of the patient, and wherein the second transducer is configured to emit energy in a direction from the external carotid artery towards the arterial nerve of the patient.

5. The device of claim 1, wherein the first and second transducers are configured to cause denervation of the carotid sinus of the patient.

6. The device of claim 1, wherein the bifurcated catheter comprises a catheter apex configured to press against a carotid bifurcation.

7. The device of claim 5, wherein the catheter apex comprises a third transducer.

8. The device of claim 6, wherein the first, second and third transducers are configured to at least partially surround a carotid sinus of the patient.

9. The device of claim 6, wherein the first, second and third transducers are configured to cause denervation of a carotid sinus of the patient.

10. The device of claim 1, wherein the first transducer is a transducer array.

11. The device of claim 10, wherein the second transducer is a transducer array, and wherein each transducer of the first transducer array corresponds to a transducer of the second transducer array that is equidistant from the catheter shaft bifurcation as its corresponding transducer in the first transducer array.

12. The device of claim 1, wherein the first catheter energy member further comprises multiple transducers.

13. The device of claim 12, wherein the second catheter energy member further comprises multiple transducers.

14. The device of claim 13, wherein at least one of the transducers of the first catheter energy member and at least one of the transducers of the second energy member emit energy simultaneously.

15. The device of claim 1, wherein the catheter shaft comprises at least one catheter lumen.

16. The device of claim 15, further comprising a first lead, wherein the first lead extends through the catheter lumen.

17. The device of claim 16, wherein the catheter lumen bifurcates at the bifurcation of the catheter shaft such that first energy member and second energy member each comprise at least one lumen.

18. The device of claim 17, further comprising a second lead wherein the first lead extends through the lumen in the first energy member and the second lead extends through the lumen in the second energy member.

19. The device of claim 18, further comprising a power connector, wherein the first lead electrically connects with the first transducer and the second lead electrically connects with the second transducer, and wherein the power connector is configured to control the amount of power emitted by the first transducer and by the second transducer.

20. A catheter, comprising:
a first catheter energy member comprising at least one transducer, wherein the first catheter energy member is configured to be deployed in an internal carotid artery;
a second catheter energy member comprising at least one transducer, wherein the second catheter energy member is configured to be deployed in an external carotid artery;
wherein the transducer of the first catheter energy member and the transducer of the second catheter energy member are configured to emit energy to a carotid sinus.

21. The device of claim 20, further comprising a catheter apex comprising a third transducer, wherein the first, second and third transducers are configured to at least partially surround a carotid sinus of the patient.

22. The device of claim 21, wherein the first, second and third transducers are configured to cause denervation of a carotid sinus of the patient.

23. The device of claim 20, further comprising a power connector configured to control the amount of energy emitted from the first and second transducers.

24. The device of claim 23, wherein the first transducer emits a different amount of energy as the second transducer.

25. A bifurcated catheter deployment kit comprising:
a sheath configured to be deployed into a common carotid of a patient;
a first guidewire;
a second guidewire; and
a bifurcated catheter, deployed into a carotid artery of the patient via the sheath, the bifurcated catheter comprising:
a first catheter energy member comprising at least one transducer, wherein the first catheter energy member is configured to be deployed, via the first guidewire, into an internal carotid artery;
a second catheter energy member comprising at least one transducer, wherein the second catheter energy member is configured to be deployed, via the second guidewire, into an external carotid artery.
